(19) United States
(12) Patent Application Publication
(10) Pub. No.: US 2015/0216442 A1
(43) Pub. Date: Aug. 6, 2015

(54) MULTILAYER COAXIAL PROBE FOR IMPEDANCE SPATIAL CONTRAST MEASUREMENT

(71) Applicants: Lev LAVY, Emek Sorek (IL); Itai HAYUT, Tel Aviv (IL)

(72) Inventors: Lev Lavy, Misgav Dov (IL); Itai Hayut, Tel Aviv (IL)

(21) Appl. No.: 14/415,736

(22) PCT Filed: Jul. 23, 2013
(86) PCT No.: PCT/IB2013/056028

§ 371 Date: Jan. 20, 2015

(30) Foreign Application Priority Data
Jul. 24, 2012 (IL) 221081

(51) Int. Cl. A61B 5/053; A61B 10/02

(52) U.S. Cl. CPC A61B 5/0536; A61B 5/0538; A61B 10/0233; A61B 2562/0209

(57) ABSTRACT

A system for spatial impedance imaging includes a multi-layer coaxial probe for spatial impedance imaging. The multi-layer coaxial probe includes: an elongated core having a distal end and a proximal end; a first coating layer wrapping around the core; a set of alternating conductive and insulating coating layers on top of said first coating layer, wherein an Nth coating layer is shorter than an N-1th coating layer beneath it. The elongated core includes a needle or other suitable elongated member.

### CROSS-REFERENCE TO RELATED APPLICATIONS

The present application claims priority and benefit from Israeli patent application number 221081, which was filed in the Israel Patent Office on Jul. 24, 2012, and which is hereby incorporated by reference in its entirety.

### FIELD

The present invention is related to dielectric measurement and positioning.

### BACKGROUND

In the medical field, a needle or a hollow needle may be used for various purposes, for example, as a probe for inspecting live tissue, for drug delivery, or for biopsy. Often, the position of the needle relative to the layers in the tissue may be important, and may affect the results of the medical procedure being performed.

Body tissue often comprises multiple layers, which may differ in their electrical properties, and particularly in their electrical impedance in response to the frequency of an applied electromagnetic signal. The impedance spectrum of a particular tissue layer may be obtained by sweeping over the electromagnetic frequency of the input signal. Analyzing the spectrum may allow determining the type of layer in which the electromagnetic signal is passing through. The spectrum may be obtained, for example, by sweeping over a frequency range, or by time-domain reflectometry (TDR) or other suitable time-domain methods.

The publication of Trebbels et al.,” Online Tissue Discrimination for Transcutaneous Needle Guidance Applications Using Broadband Impedance Spectroscopy” (IEEE Transactions On Biomedical Engineering, Vol. 59, No. 2, pages 494-503, February 2012) describes a system architecture for measuring impedance spectra of a biological tissue close to the tip of a hollow needle. The measurement is performed online using fast broadband chirp signals. The time domain measurement raw data are transformed into the transfer function of the tissue in frequency domain. Correlation technique is used to analyze the characteristic shape of the derived tissue transfer function with respect to known “library functions” for different types of tissue derived in earlier experiments. Based on the resulting correlation coefficients, the type of tissue is determined.

The publication of Kalvoy et al., “Impedance-based tissue discrimination for needle guidance” (Physiol. Meas. Vol. 30, No. 2, pages 129-140, 2009) attempts to discriminate between muscle and fat (or subdermis) for purposes of drug administration, by interpreting electrode polarization impedance (EPI) at low frequencies.

U.S. Pat. No. 6,337,994 to Stoianovici et al. is entitled “Surgical needle probe for electrical impedance measurements”, and describes an electrical impedance probe that includes a surgical needle. The probe is a two-part trocar needle designed to acquire impedance measurements at its tip. The impedance measurements are representative of local properties of a biological substance at the needle tip.

U.S. Pat. No. 5,335,668 to Nardella is entitled Diagnostic impedance measuring system for an insufflation needle”, and describes an elongate tissue-penetrating probe member with a plurality of axially spaced reference electrodes disposed about a distal portion of the probe. The reference electrodes measure the impedance of the biological tissue adjacent each electrode.

U.S. Pat. No. 6,096,035 to Sodhi et al. is entitled “Multipolar transmural probe”, and describes a needle-like probe for use in electrical potential sensing and RF ablation of tissue. The probe has an elongated body which comprises two or more electrodes separated and spaced apart from each other by insulative material. Each electrode is capable of delivering RF energy to the tissue surrounding the electrode and sensing the electrical potential of the tissue.

### SUMMARY

The present invention provides devices and methods for dielectric measurement and positioning, and may enable impedance-contrast sensing of different dielectric layers. In accordance with the present invention, a positioning device may utilize a signal computed based on the measured frequency-dependent capacitance and resistance (i.e., impedance) of a sample or tissue surrounding multiple parts of a needle or probe. The measurements may assist the positioning of the needle or probe, and may enable accurate insertion of the needle or probe into the desired dielectric layer, while measuring the dielectric properties of the layers surrounding the needle or probe.

In accordance with the present invention, a system for spatial impedance imaging comprises a multi-layer coaxial probe for spatial impedance imaging. The probe comprises: an elongated core having a distal end and a proximal end; a first coating layer wrapping around the core; a set of alternating conductive and insulating coating layers on top of said first coating layer, wherein an Nth coating layer is shorter than an N-1th coating layer beneath it.

In some embodiments, each coating layer begins at a first distance from the proximal end, and ends at a second distance from the distal end; and values of the first distance and the second distance increase for external coating layers relative to internal coating layers.

In some embodiments, each coating layer has a thickness of approximately 0.5 micron to 50 micron.

In some embodiments, a tip of the probe is round.

In some embodiments, a tip of the probe is tapered.

In some embodiments, at least one of the coating layers comprises a partial coating layer that provides partial coating to a layer underneath said partial coating layer.

In some embodiments, at least one of the coating layers comprises a thin spiraling wire.

In some embodiments, at least one of the coating layers comprises a thin helix-shaped wire.

In some embodiments, edges of the coating layers are distributed along a sensing area adjacent to a tip of said probe in accordance with a desired spatial resolution.

In some embodiments, the core comprises a hollow needle to enable at least one of: injection of a fluid; delivery of a drug; extraction of a biopsy.

In some embodiments, the core comprises a pre-fabricated medical needle that is subsequently coated with said alternating coating layers.

In some embodiments, the system further comprises: an electric signal source to provide an electric signal to each pair of conductive layers; and an electric signal measurement unit to measure impedance differences between pairs of conductive layers.

In some embodiments, the system further comprises: a processing module to determine a location of a tip of said multi-layer coaxial probe within a sampled item, based on said measured impedance differences.

In some embodiments, said pairs of conductive layers comprise at least one pair of non-neighboring conductive layers.

In some embodiments, said pairs of conductive layers comprise at least one pair of neighboring conductive layers.

In some embodiments, the system further comprises: an output unit to provide to a user of the probe a real-time indication of a current location of a tip of the probe; wherein the real-time indication comprises at least one of: an audible indication, a vibrating indication, a visual indication.

In some embodiments, the probe is connected to a socket comprising a set of electrodes to receive an electromagnetic signal via electric wires from a signal source.

In some embodiments, the probe comprises a needle selected from the group consisting of: an epidural needle, a biopsy needle, a drug delivery needle, a cosmetic needle, an intravenous (IV) needle, a draining needle.

In some embodiments, the set of alternating coating layers coat a tip of said probe to form therein one or more sensing points.

In some embodiments, the conductive layers are formed of titanium, and the insulating layers are formed of glass.

In some embodiments, the probe is directly connected to a signal generator and a measuring sub-system.

In some embodiments, the probe is indirectly connected via a socket, to a signal generator and a measuring sub-system.

In some embodiments, the socket comprises a relay module to enable switching among electrodes of the probes.

In some embodiments, the socket comprises: a battery, and a wireless transmitter to wirelessly transmit electric signals.

In some embodiments, the elongated core comprises an elongated needle-like member.

In some embodiments, the multi-layer coaxial probe is a multi-layer quasi-coaxial probe.

In some embodiments, the multi-layer coaxial probe is non-symmetric along the long dimension of the elongated core.

In some embodiments, the elongated probe comprises a needle.

In some embodiments, the elongated probe comprises a needle having a non-circular cross-section.

In some embodiments, the elongated probe comprises a needle having a square-shaped cross-section.

In some embodiments, the elongated probe comprises a needle having a triangular cross-section.

In some embodiments, the elongated probe comprises a thin sharp blade.

In some embodiments, each coating layer has a thickness in the range of 0.1 percent to 3 percent of a thickness of the elongated probe.

In some embodiments, the elongated probe comprises a heat-transfer unit to enable selective heating of a location of interest.

In some embodiments, the elongated probe comprises an electrical energy-transfer unit to enable selective electric stimulation of a location of interest.

In some embodiments, at least one of the conductive coating layers comprises: a first layer of titanium, having thickness of 1 to 100 nanometers; covered by a layer of a metal other than titanium, having thickness of 500 to 5,000 nanometers, said metal having greater electrical conductivity than titanium; covered by a second layer of titanium, having thickness of 1 to 100 nanometers. Optionally, at least one of the metals is bio-compatible.

In some embodiments, wherein at least one of the conductive coating layers comprises: a first layer formed of a first metal, having thickness of 1 to 300 nanometers; covered by a second layer formed of a second metal, having thickness of 500 to 5,000 nanometers, said second metal having greater electrical conductivity than said first metal; covered by a third layer formed of said first metal, having thickness of 1 to 300 nanometers. Optionally, at least one of the metals is bio-compatible.

In some embodiments, at least one of the conductive coating layers comprises: a first layer of titanium; covered by a layer of a metal other than titanium, said metal having greater electrical conductivity than titanium; covered by a second layer of titanium.

In some embodiments at least one of the conductive coating layers comprises: a first layer of titanium, having thickness of 1 to 250 nanometers; covered by a layer of a metal other than titanium, having thickness of 500 to 5,000 nanometers, said metal having greater electrical conductivity than titanium; covered by a second layer of titanium, having thickness of 1 to 250 nanometers.

In some embodiments, an outmost coating layer of said multi-layer coaxial probe is more rigid than other coating layers of said multi-layer coaxial probe.

In some embodiments, an outmost coating layer of said multi-layer coaxial probe is formed of a bio-compatible material.

In some embodiments, an outmost coating layer of said multi-layer coaxial probe is formed of a hydrophobic material.

In some embodiments, an outmost coating layer of said multi-layer coaxial probe is formed of a hydrophilic material.

In some embodiments, an outmost coating layer of said multi-layer coaxial probe comprises a gold layer.

In some embodiments, an outmost coating layer of said multi-layer coaxial probe comprises a gold layer applied by electro-plating.

In some embodiments, at least one of the insulating coating layers is formed of a material selected from the group consisting of: glass, plastic, resin, gum.

In some embodiments, at least one pair of an adjacent conductive coating layer and insulating coating layer comprises: a conductive coating layer formed of a metal; and an insulating coating layer formed of said metal that was subjected to oxidation.

The present invention may allow other and/or additional benefits and advantages.

### BRIEF DESCRIPTION OF THE DRAWINGS

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. The figures are listed below.

FIG. 1 is a schematic illustration of a multilayered coaxial probe, in accordance with some demonstrative embodiments of the present invention;

FIG. 2 is a schematic illustration of a probe assembly, in accordance with some demonstrative embodiments of the present invention;

FIG. 3 is an exploded view of a probe assembly, in accordance with some demonstrative embodiments of the present invention;

FIG. 4 is a schematic cross-section view of a sensing edge of a probe, in accordance with some demonstrative embodiments of the present invention;

FIG. 5 is a schematic illustration of a sensing edge of a probe, penetrating into a substance having multiple layers, in accordance with some demonstrative embodiments of the present invention;

FIG. 6 is a flow-chart of a method in accordance with some demonstrative embodiments of the present invention;

FIGS. 7A-7D are schematic illustrations of a multi-layer coaxial probe, in accordance with the present invention; and

FIG. 8 is a schematic illustration of a multi-layer coaxial probe, in accordance with the present invention.

### DETAILED DESCRIPTION OF THE PRESENT INVENTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Applicants have realized that an important aspect in needle insertion may be transmission of an electromagnetic signal through the edge of the needle, to enable detection of the next layer that is about to be penetrated, prior to penetrating it. When the needle passes through multiple layers, it may become complicated to process the electromagnetic signal and detect in which layer the needle is present, and it is difficult to estimate the distance that the needle passed in each layer.

The present invention may utilize impedance spectroscopy for medical needs, and may provide a robust solution to the problem of spatial detection of a multilayer sample by introducing spatially separated electrodes along the penetrating needle.

The present invention differs from conventional devices, for example, in the information that the needle or probe extracts, as well as in the structure and function of such needle or probe. A conventional probe aims to set the penetration depth of the probe and the change in the impedance by using electrodes that are located in the tip of the probe. Applicants have realized that such conventional probes require calibration of the electrodes to known samples, and do not provide data regarding sample properties that are along the needle (as opposed to the needle tip).

Applicants have further realized that conventional methods utilize absolute values of the electrical properties of the sample. Such absolute values obtained from a single point may be subjected to errors, for example, due to temperature change, liquid leakage, chemical reaction on the electrodes, or other factors that may bias the measurements.

Applicants have also realized that conventional methods may fail or may be inaccurate when the medical procedure requires positioning of the needle relative to the layers sampled in or at a specific depth. Some conventional implementations may utilize a highly-precise robotic arm for such needle placement, thereby rendering the implementation both costly and complex to operate.

The present invention may include a device for efficient spatial probing. The device may utilize (or may operate in conjunction with) any suitable needle as a probe, and may be able to extract signal information in all relevant frequencies (including, particularly, high frequencies. The present invention may utilize real-time differential impedance measurement with multiple sensing points along the probe with (or providing) DC to RF spectra. The device may utilize coaxial, multilayer electrode waveguides, enabling signal isolation and noise reduction, thereby allowing dielectric one-dimensional imaging along the needle probe.

The present invention may provide a dielectric measurement and positioning device which may utilize impedance-contrast sensing of different dielectric layers of the sampled (or probed) substance or item. The device may utilize a signal computed from the measured frequency-dependent capacitance and resistance (i.e., impedance) of the sample or tissue surrounding different parts of the device. This may assist the positioning of the device and may allow accurate insertion of the instrument into the desired dielectric layer, while measuring the dielectric properties of the layers surrounding the device (or surrounding the tip of the device).

The probe of the present invention may allow spatial sensing of a multilayer sample by using electric measurement in real time. For example, an electromagnetic signal that is returned by the probe may be analyzed in real time in order to map the dielectric contrast between the sample layers, and to allow estimation of the distance from the tip of the probing device to the next layer that is about to be reached or penetrated in a subsequent step of the insertion process (and prior to actual subsequent reach or penetration).

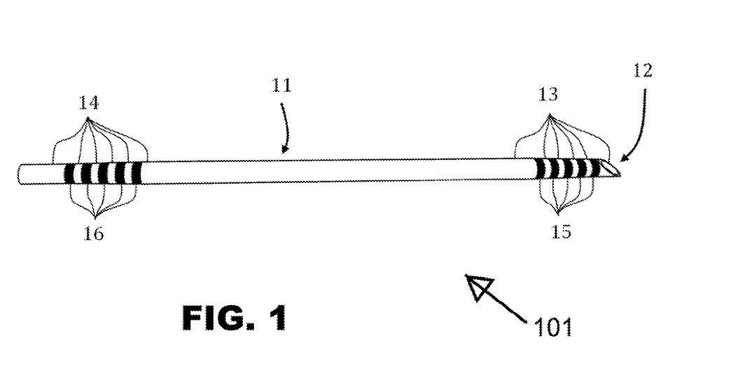

Reference is made to FIG. 1, which is a schematic illustration of a multilayered coaxial probe 101, in accordance with some demonstrative embodiments of the present invention. Probe 101 may comprise an elongated rod-like member, for example, a needle 11 or other suitable probing element, thin element, sharp element, tapered element, or the like. Optionally, needle 11 may be hollow or may include an elongated tunnel or cavity 12, in order to allow, for example, suction or removal or withdrawal of substance(s), and/or insertion or injection or delivery or implantation of substance(s).

Needle 11 may be formed of conductive material(s), or may be generally coated with an electrically conductive material. Particular portions or regions of the external layer of needle 11 may be coated with (or formed from) resistive or insulating or non-conductive material(s). In a demonstrative implementation, regions 13-14 may have an electrically conductive coating; whereas regions 15-16 may have an electrically resistive coating and may be generally ring-shaped or band-shaped. As demonstrated, conductive and resistive regions may be located alternately, and may be grouped in batches or groups (e.g., a first group located at or near the tip area of the probe, and a second group located at or near the socket area of the probe); for example, resistive regions 15 grouped as a first group, and resistive regions 16 grouped as a second group. The location or relative location of regions 13-16, the number regions included in regions 13-16, and the grouping of regions among regions 13-16, may be determined in order to provide a desired spatial resolution.

Needle 11 may be inserted into a sample, for example, a substance, a body organ or a patient, or a multi-layered object. During the insertion process, electric signal source 104 may provide an electric signal, for example, to each pair of conductive regions. This may allow measurement of the differences in electric impedance between the materials surrounding different parts of needle 11.

Probe 101 may comprise conductive and insulating layer pairs, which may be formed by coating needle 11 to achieve a multi-coaxial transmission line. The edge of each additional layer may start and end closer to the center of needle 11, to enable spatial measurement. Each layer (e.g., conductive layer or isolating layer) may end at an increasing distance from the distal end of needle 11; and this structure may enable sensing of the surrounding sample at different spatial point(s). Each set of electrodes may be spread away from the distal end, for example, at equal distances (e.g., to allow for efficient calibration of the electrical signal) or with different distances for different sensing area sizes.

In a demonstrative example, needle 11 may comprise an 18G biopsy needle, having length of 3.5 inches or approximately 10 centimeters. Accordingly, probe 101 may be structured to have ten sensing points, by including 20 or 21 alternating layers (isolating layer; conductive layer; isolating later, and so forth) around the “core” needle 11. The first, most-internal, coating layer that directly touches needle 11 may be an isolating layer, and may cover needle 11 starting after 1 millimeter and ending 0.5 millimeter before the tip; whereas consecutive coating layers may start and end in a similar manner after and before the previous layer starting and ending points. This structure may allow approximately 1 millimeter of spatial resolution along 1 centimeter of sensing area at the distal end and 2 centimeters for connector or interface part(s). In some embodiments, optionally, the most outer layer may be an insulating layer or a non-conductive layer; and optionally, may be a finely smoothed layer in order to allow smooth insertion and removal of the probe; other type(s) of outer layer may be used, for example, a dedicated outer layer, a hydrophobic outer layer, a hydrophilic outer layer, an anti-bacterial outer layer, an anti-microbial outer layer, a bio-compatible outer layer, or the like.

Optionally, the fabrication process may be performed at an angle (e.g., by using sputtering), thereby providing to the distal end an angle or a tapering for smooth penetration of the sample. Alternatively, multi-step fabrication may be used (e.g., manufacturing each layer separately), to similarly provide a smooth ending or a tapered ending. Additionally or alternatively, the manufacturing process may comprise filing or etching or cutting the surface or edge or ending of needle 11 after fabrication, and/or heating needle 11 (e.g., at temperature which may be close to the melting point of the isolating layer). Other suitable manufacturing techniques may be used.

In a first demonstrative implementation, needle 11 may comprise a conductive core, which may be coated by an insulating layer, then by a conductive layer, then by an insulating layer, then by a conductive layer, and so forth.

In a second demonstrative implementation, needle 11 may comprise a insulating core, which may be coated by a conductive layer, then by an insulating layer, then by a conductive layer, then by an insulating layer, and so forth.

In another demonstrative implementation, a needle may be approximately 4 centimeters long, and may have a diameter of 2 millimeters and a desired spatial resolution of 1 millimeter at the last centimeter of the probe. Accordingly, thickness of each layer may be approximately 10 microns in order to avoid distortion of the needle shape while maintaining good conductive and insulating properties. The layers edges distribution may be equal along the sensing area and along the connection side to the socket.

The multi-layer coating scheme that utilizes alternate conducting/insulating layers may allow spatial resolution of at least two points along needle 11, to enable contrast measurement. The resolution may be determined by the axial distance between conductive and insulating layer pairs in the sensing edge of probe 101. The number of probing points along needle 11 may be determined by, or may correspond to, the number of conducting and insulating layer pairs.

The radius of electrodes may change as additional layers are added, and thus the thickness of the layers may be determined in order to ensure that the multiple layers, which act as electrodes, may have identical (or generally similar) electrical characteristics, for simplifying the signal analysis.

In some embodiments, for example, conductive layers may be formed of titanium; and insulating layers may be formed of glass. Other suitable materials or metals may be used, for example, silver, aluminum, gold, iron, or the like.

In a demonstrative implementation, a conductive layer may be formed as a composite material, or by using multiple composite materials; or as multiple metallic layers, for example, as follows: a thin layer of approximately 1 to 200 nanometers of titanium, covered by a thicker layer of approximately 500 to 5,000 nanometers (or, approximately 0.5 to 5 microns) of a metal that conducts electricity better than titanium (such as, for example, aluminum), and covered by a thin layer of approximately 1 to 200 nanometers of titanium. In such implementation, the electrode may be bio-compatible, since titanium is bio-compatible with the tissue; whereas the electrode may still have high conductivity due to the utilization of aluminum. Additionally, the titanium may serve other purpose(s), for example, gluing or bonding the glass layer to the electrode, since titanium may bond well to both glass and metal. In some implementations, the titanium layer may be extremely thin or super-thin, or may be a single atomic layer of titanium atoms (e.g., having layer thickness of approximately 1 nanometer, or in case of single layer 0.15 nanometer). In some implementations, the titanium layer may coat or cover all, or substantially all, or most, of the aluminum layer (e.g., by slightly moving the probe or the mask in the manufacturing process).

In some implementations, the electrodes may be coated with gold, for example, in an electro-plating process which may be performed during and/or subsequently to the formation of the layers. Accordingly, various suitable metals may be used (e.g., aluminum, silver, copper), as the gold electro-plating may provide bio-compatibility and may protect the inner metal(s) from oxidation.

Various materials may be used to form the non-conductive layers, for example, glass, plastic material(s), resin, gum. For example, glass may be used in order to allow vaporization (or other process of placement) of titanium on the glass, as a transition layer that may be used for gluing or bonding a metal layer. It is noted that each layer may wrap around, or engulf, some or most of the layer beneath it, and this structure may contribute to bonding of layers and may prevent separation of layers or gaps between layers.

In some implementations, a metal layer (e.g., an iron layer) may be used, and may be subject to intentional and selective oxidation up to a pre-defined depth; thereby converting one layer (e.g., an iron layer) to become a conductive layer coated by an insulating layer.

In some embodiments, the most outer layer (or, the outmost layer or external layer) may be thicker (or significantly thicker) than other layers, in order to render the probe more rigid and/or to protect the other (internal) layers, without covering and without obstructing the sensing regions. Some embodiments may utilize a material that shrinks upon getting cold, thereby wrapping tightly around the core or around internal layer(s) and contributing to the bonding among layers. Some embodiments may utilize a thin protective layer, for example, all along the probe, including (optionally) protection of the sensing regions.

For example, to maintain the same electrical properties of the probes, one or more equations or formulae may be used. In some embodiments, measuring may utilize low frequencies (e.g., wavelength is greater than the size of the system), and thus the system may utilize lumped element approximation. Optionally, the implementation may utilize an assumption that the change in the overall diameter of the needle is relatively small, and the diameter of the needle is much greater than a layer thickness.

For example, the following equations may be used:

R=ρ·L/Aᵣ=ρ·L/(π·d·h_{electrode}) (1)

C=ε·A_{c}/h_{c}=ε·L·π·d/hᵢₙₛᵤₗₐₜₒᵣ (2)

In Equations (1) and (2), R may be the total electrical resistance of an electrode (e.g., the electric resistance of the electrode when measuring by using a first port attached to the distal end and another port attached to the proximal end); C may be the capacity between two electrodes; ρ may be resistivity of the electrode material; L may be the length of the electrode; ε may be permittivity; hᵢₙₛᵤₗₐₜₒᵣ may be the height (or thickness) of an insulating layer; h_{electrode} may be the height (or thickness) of the electrode layer; Aᵣ may be the cross sectional area of the electrode; and A_{c} may be the surface size of the electrode.

Some implementations may utilize one or more constraints, for example, constraints that subsequent sensing point self-resistance and self-capacitance be the same as those of the current sensing point; for example, expressed in the following equations:

Cₙ₊₁=Cₙ (3)

Rₙ₊₁=Rₙ (4)

Accordingly, the following functions (e.g., recursive functions) may be utilized:

h_{electrode,n+1}=h_{electrode,n}(L₀−Lₙ₊₁)/(L₀−Lₙ) (5)

h_{insulator,n+1}=h_{insulator,n}(L₀−Lₙ₊₁)/(L₀−Lₙ) (6)

In case of constant spacing between the electrodes:

Lₙ=n·dl (7)

In the above equations, n may be the index of the sensing point; and dl may be the desired change in the electrode length per sensing point. Lₙ is the length of the n electrode; and L₀ is the length of the first electrode.

If dl changes for each layer, then Equations (5) and (6) may utilize, instead of Lₙ=n·dl of equation (7), a sum of the lengths.

In a demonstrative example, the initial properties of the needle may be: L₀=10 centimeters, h_{electrode,0}=1 micron, h_{insulator,0}=10 micron, dl=1.5 millimeters, and the desired number of sensing points may be 10. Accordingly, the height values of the conductive coating layers (denoted h_{electrode}) may be: 1 micron, 0.984 micron, 0.969 micron, 0.953 micron, 0.938 micron, 0.922 micron, 0.907 micron, 0.892 micron, 0.876 micron, 0.861 micron; and the height values of insulating layers (denoted hᵢₙₛᵤₗₐₜₒᵣ, and equaling ten times the corresponding value of h_{electrode}) may be: 10 micron, 9.84 micron, 9.69 micron, 9.53 micron, 9.38 micron, 9.22 micron, 9.07 micron, 8.92 micron, 8.76 micron, 8.61 micron.

Optionally, an electric signal from the probe electrodes may be calibrated (e.g., once, or in multiple iterations), relative to each other, to enable robust measurement. For example, the calibration process may be performed by measuring the measurement device self-impedance, as well as the cable self-impedance, the connector self-impedance, and the needle self-impedance. The calibration process may be repeated for each one of the sensing points, and may be performed with a sample of air, short circuit and matched impedance. The calibration may be performed automatically (e.g., at the fabrication line), particularly if the needle is kept at room temperature; optionally, vacuum environment may be used for keeping the needle from changing its electrical properties. The calibration process may also be performed with samples having similar properties to the sample to be measured (e.g., body tissue, saline), to enhance the sensitivity in the desired range of electrical properties. The calibration may be performed per needle, or per particular needle structure, or for a batch of generally-identical needles.

In order to measure impedance between each of the conductive layer pairs, one side of needle 11 may be connected to a socket containing a set of electrical contacts. The socket may further be connected (e.g., via wires or other suitable means for electrical transmission) to a measuring device. Optionally, a relay element (or other adapter or interface) may be used in conjunction with the electric socket, to reduce or minimize the number of connective wires between the electric socket and the measuring device (or its cable).

The edges of the coating layers may be spread along needle 11 to achieve a desired spatial sensitivity on the probing edge of needle 11. At the edge of needle 11 that connects to the electrical socket, coating edges may be spread evenly, for example, to simplify or facilitate the assembly or the connection process.

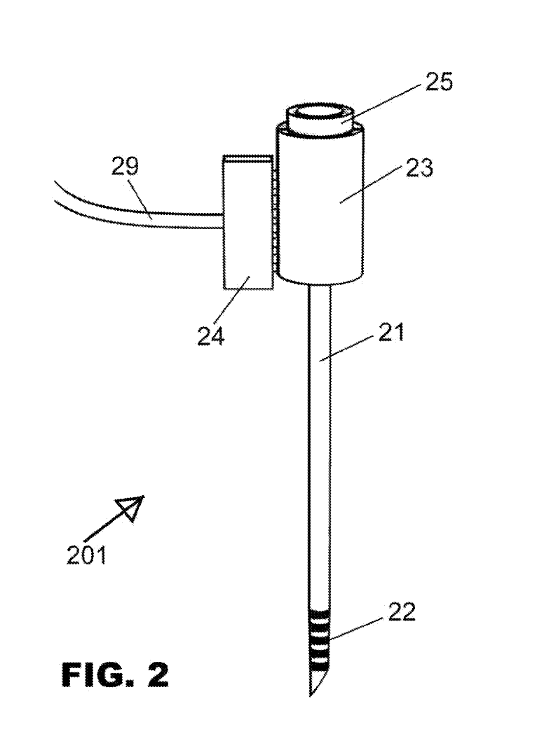

Reference is made to FIG. 2, which is a schematic illustration of a probe assembly 200, in accordance with some demonstrative embodiments of the present invention. Probe assembly 200 may comprise a probe 21, which may be generally similar to probe 101 of FIG. 1. As demonstrated in FIG. 2, a first edge of probe 21 may be a sensing edge 22; whereas a second edge of probe 21 may be held by (or may be inserted into) an electrical socket connection 23, which may be connected to a measurement system cable 29 via an optional interface or adapter 24. Optionally, a syringe socket 25 may be connected or attached to probe 21, particularly in implementations in which probe 22 is hollow or comprises a hollow needle.

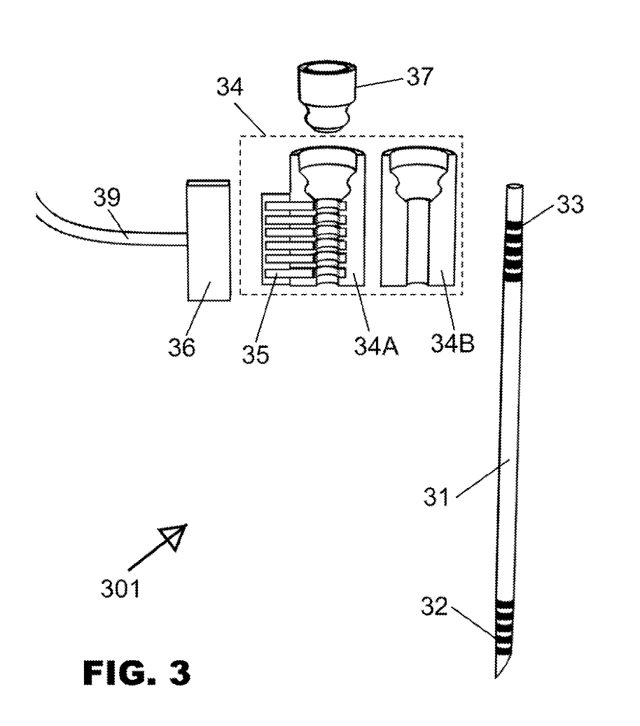

Electrical socket connection 25 may connect probe 21 to a measurement device, in which the electrical signal may be created and transmitted to electrodes of probe 21 (shown in FIG. 3). The impedance may be calculated using a scope that samples the electrical signal. Measurement data may be stored in a short-term or long-term memory unit or storage unit (e.g., Flash memory, hard disk drive, buffer(s), RAM units, or the like). Data may be analyzed by using a local or remote processing unit, processor, controller, Integrated Circuit (IC), system on a chip (SOC), workstation, portable electronic device, smartphone, tablet, laptop, general-purpose computing device, or other suitable device. Optionally, data processing may be performed live or in real-time by a server which may provide processing services to multiple or many units, based on a subscription fee, a pay-per-use fee, a pay-per-time-period subscription fee, or other suitable methods.

Optionally, an electrical model may be predefined for the sample (or type of sample) being probed and/or for the particular probe being used, and the electrical properties of the sample may be obtained by calculations. The measurement may be repeated with each pair of electrodes in order to achieve spatial measurement. Optionally, measurement(s) may be performed between conductive layers that are not adjacent to each other.

In a demonstrative implementation in the medical field, the probing device may be used where there is a need to inject or extract once the needle reaches a desired position or location. The position may be accurately set by using the spatial probe measurement that may be done in real time, e.g., while the probe is being gradually inserted into the body organ. Optionally, the needle may be hollow and may be attached to a socket allowing connection to a syringe.

Reference is made to FIG. 3, which is an exploded view of a probe assembly 301 in accordance with the present invention. For example, a probe 31 may comprise a sensing edge 32 and a connection edge 33. Connection edge 33 may be connected to an electrical connection socket 34, for example, comprising a first side 34A having multiple contacts or electrodes 35 and a tightly-gripping cover side 34B. A measurement system cable 39 (e.g., a coaxial cable) may be connected to the electrical connection socket 34, directly or via an optional adapter or interface 36 (e.g., able to receive electrical signal and to provide electrical feedback). Optionally, a syringe socket may be included in probe assembly 301.

Optionally, multiple wires or cables may be used, for example, utilizing a separate cable or wire for each conductive layer; with a relay unit located externally to the probing device. In some implementations, the use of the relay unit may be optional, and simultaneous or concurrent measurement of multiple electrodes (or all electrodes) may be used.

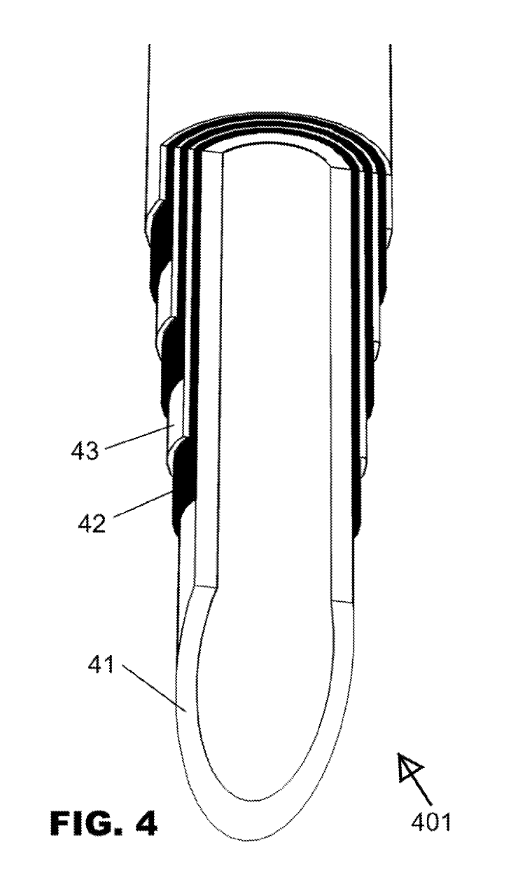

Reference is made to FIG. 4, which is a schematic cross-section view of a sensing edge 401 of a probe in accordance with the present invention. As demonstrated, a needle core 41 may be surrounded or coated by multiple layers of alternating conductivity/resistance properties, for example, alternating conductive portions 43 and insulating portions 42. In some implementations, sensing edge 401 and its coating layers may have a gradually-narrowing structure, a wedge or tapered structure, or a gradually-thinning structure.

In accordance with the present invention, needle core 41 may be used as the base of the probe, on which pairs of conductive layers 43 and insulating layers 42 may be alternately coated. Each pair of conductive layers 43 functions as an additional set of probing electrodes.

The coating of the layer may be thin relative to the needle radius, to keep the electrode pairs with the same impedance as well as for maintaining a needle shape. The thickness of the layers may vary slightly, for example, in order to compensate the change in the radius of the layers to maintain the electrodes impedance. The matching of electrodes impedances may be important in order to simplify the calibration, or even to obviate the need for calibration.

The processing algorithm of the measurement results may utilize a model of the sample layers to enable greater precision and to more accurately calculate the next layer position relative to the needle tip electrode before the needle actually punctures or penetrates the next layer. The sampled layers position and properties may be presented in real time, using a display unit or monitor or a smaller display which may be in proximity to the probing device. Audio speaker(s) may be used to generate an audible signal, for example, when a particular layer is reached, or when the needle tip moves (or is about to move) from layer to layer.

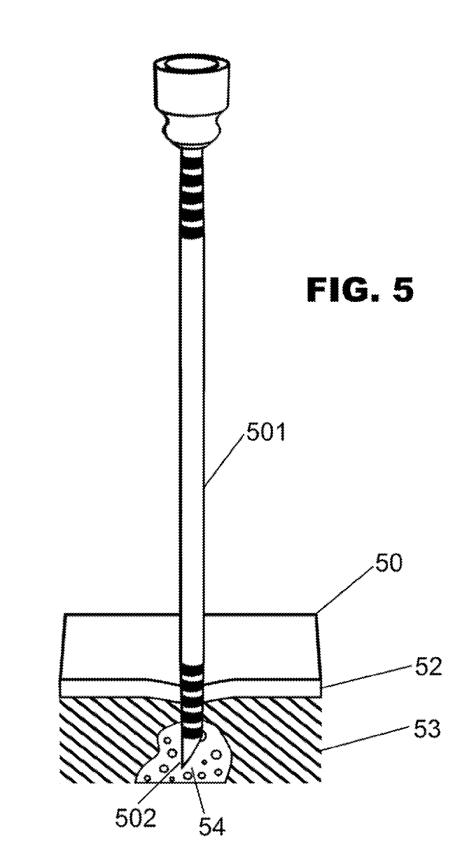

FIG. 5 is a schematic illustration of a sensing edge 502 of a probe 501, penetrating into a substance 50 having two (or more) layers 52-53, in accordance with some demonstrative embodiments of the present invention. For example, two most-distal sensing points are within the inner tissue or sample; the next sensing point from the distal end is in the skin or outer sample; the most outer sensing point is in the air. The impedance contrast image may provide indication where the needle position is relative to each layer. In some implementations, the desired injection or extraction position may be in-between layers of the sample (e.g., intra-layer location), and the probe may provide the knowledge of where to move the needle to without the need to know the exact properties of the sample. Since the measurement signal of each sensing points may be compared to each other, high sensitivity may be obtained as well as real-time calibration to avoid drifts. In some embodiments, probe 501 may be inserted such that its tip or sensing edge 502 reaches exactly a particular region 54, which may be a tumor, or a cancer tissue, or a region that has to be treated, or a region for biopsy or extraction, or a region for injection or for drug delivery, or the like.

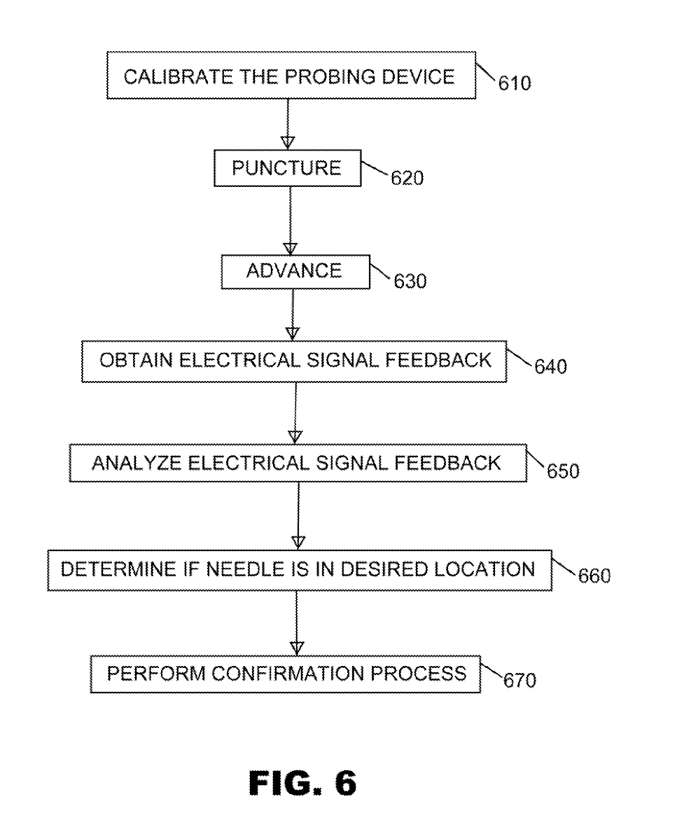

Reference is made to FIG. 6, which is a flow-chart of a method in accordance with some demonstrative embodiments of the present invention. The method may be used, for example, in conjunction with a probing device or probing system as described herein. The method may be used in-vivo, or in conjunction with treating a patient; or may be used ex-vivo; or may be used externally to a human body, or without any relation to treating the human body. The method may be used for non-medical goals, for example, for soil diagnosis, for tree or plant or fruit diagnosis, for food diagnosis; or may be used for treatment of pets or animals.

Optionally, the method may comprise calibrating the probing device (block 610), or otherwise establishing baseline measurement value(s).

The method may comprise puncturing (block 620) and advancing (block 630), or other operations to achieve gradual insertion of the probing device into the sample being probed. The inserting may be in discrete stages (e.g., advancing one millimeter at a time, then pausing, then advancing again, then pausing, and so forth); or may be in a continuous advancing motion.

The method may comprise obtaining electrical signal feedback (block 640), for example, substantially continuously or in discrete time intervals (e.g., every 0.1 second).

The method may comprise analyzing the electrical signal feedback (block 650), for example, to determine current position or location of the tip or edge of the probing needle.

The method may further comprise, for example, determining whether or not the needle tip is located in a desired destination location (block 660). If the checking result is negative, then the method may comprise repeating the steps of blocks 630 and onward. Alternatively, if the checking result is positive, then the method may proceed with the steps of block 670 and onward.

Once the needle tip reaches its destination location, the method may further perform an optional confirmation process (block 670); for example, Computed Tomography (CT) scanning in case of a medical procedure and the use of needle sensing may reduce the radiation exposure for the confirmation step.

The present invention may enable a probe or needle to be guided to a specific layer and/or depth in the sample (or to an intra-layer location), as well as measuring and characterizing the properties of the sample or of particular layer(s) thereof.

In accordance with the present invention, the needle or probe may be fabricated by spattering, such that a substance may be heated (e.g., in a vacuum environment) and spattered upon the needle core. Optionally, a mechanical mask may be used. Optionally, to ensure the continuity and homogeneity of the coating layers, the needle core (or the needle as it is being formed) may spin along or rotate its axis during the spattering process. The process may be repeated for each coating layer with the appropriate conductive or insulating material(s).

The outer layer of the probe or needle may be hydrophobic (e.g., to avoid the accumulation of a water layer, and/or to enable easy penetration in tissue) or hydrophilic, depending on the desired application of the probe.

Depending on the application of the probe, the length and diameter of the probe may be determined The preferred electrical signal frequency bandwidth may be set according to the geometry of the particular probe. For example, the main frequency may be on a self resonant of the needle, in order to achieve enhanced sensitivity. The self resonant may correspond to half wave-length of the probe, and may be tuned according to the insulated layer and the sample electrical properties. For example, when probing human tissue, the needle may have a length of a few centimeters, and a possible frequency of the electrical signal may be in the range of approximately 0.1 to 1 GHz, or approximately 1 MHz to 10 GHz, or approximately 1 MHz (or DC) to 18 GHz, or other suitable frequency or frequency-range. For example, a frequency of approximately 1 GHz may be used in conjunction with a needle or probe of several centimeters; whereas a frequency of approximately 18 GHz may be used for a millimeter-needle utilized for cosmetic drug delivery to a desired sub-skin layer. In an implementation directed at, for example, probing soil for moisture (e.g., for agricultural purposes), the length of the probe may be a few meters, and a lower frequency may be used, for example, in the range of approximately 1 to 10 MHz.

The present invention may provide a multi-layer coaxial probe device, which may be based on coating a core needle from substantially all its sides with alternating conductive and isolating layers, that end on the distal part in increasing distance from the edge, thereby enabling spatial impedance imaging.

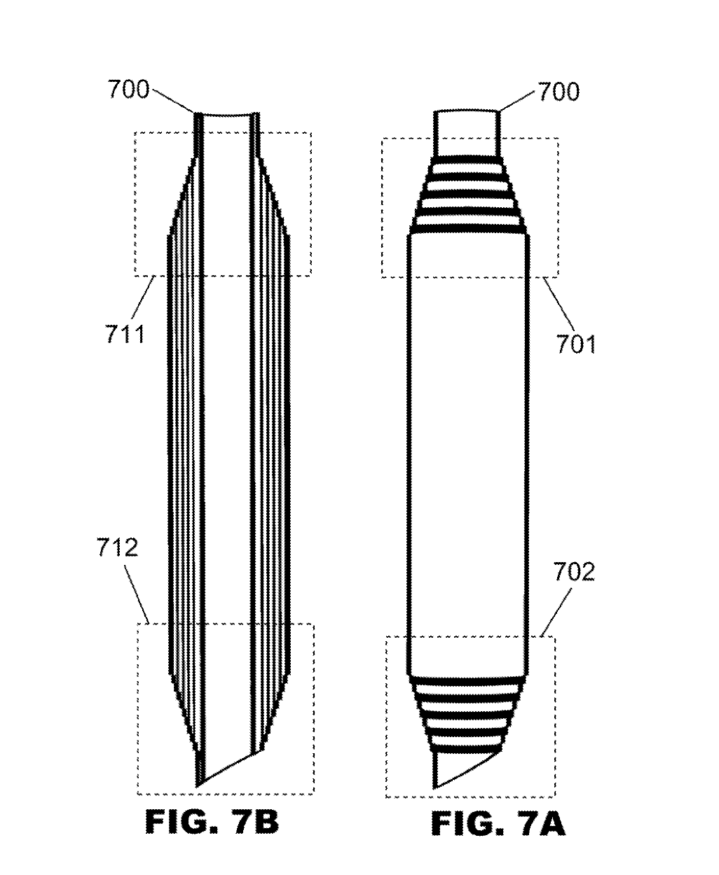

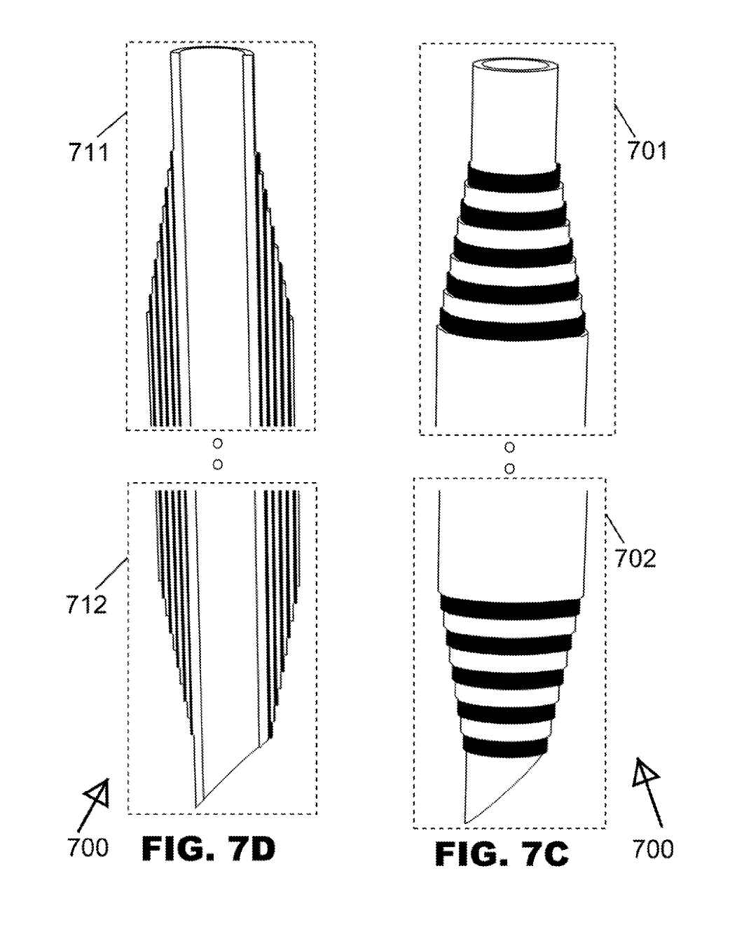

Reference is made to FIG. 7A, which is a schematic illustration of a multi-layer coaxial probe 700 in accordance with the present invention, demonstrating alternating coating layers (e.g., conductive, insulating, conductive, insulating, and so forth). Reference is further made to FIG. 7B, which is a schematic illustration of a cross-section view of probe 700 of FIG. 7A.

Reference is further made to FIG. 7C, which is a schematic illustration of probe 700 of FIG. 7A, showing a portion 701 in a greater detail, and also showing a portion 702 in greater detail.

Reference is further made to FIG. 7D, which is a schematic illustration of probe 700 of FIG. 7B, showing a portion 711 in a greater detail, and also showing a portion 712 in greater detail.

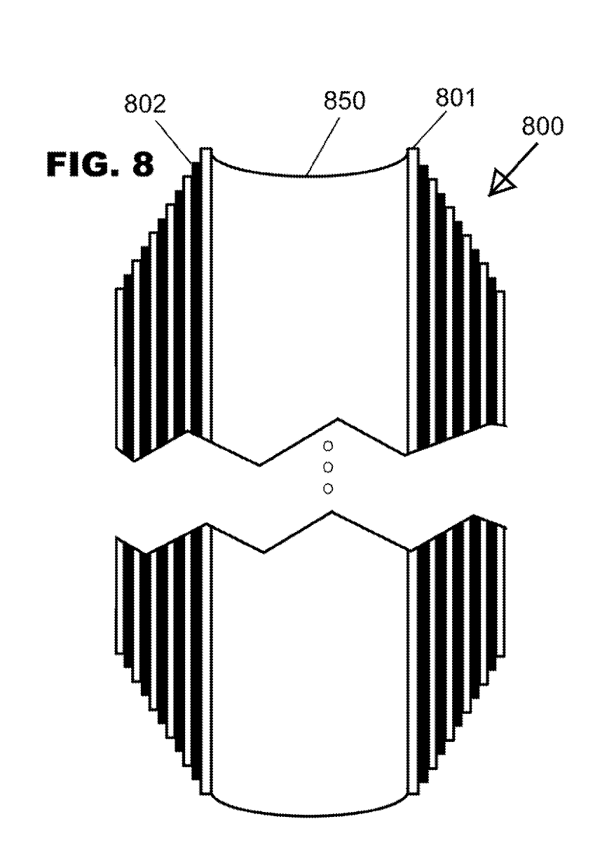

Reference is also made to FIG. 8, which is a schematic illustration of a multi-layer coaxial probe 800 in accordance with the present invention, demonstrating a core 850 surrounded by alternating coating layers (e.g., insulating layer 801, conductive layer 802, insulating layer 801, conductive layer 802, and so forth).

A micro-structure may be used, in order to achieve minimal interference of the equipment (e.g., particularly for medical purposes); the needle may be thin and smooth, and each coating layer may be in the range of several microns height (or thick). In some implementations, for example, a thickness or height value of each conductive coating layer, or a thickness or height value of each isolating coating layer, may be in the range of 0.1 micron to 20 micron; or in the range of 0.5 micron to 15 micron; or in the range of 1 micron to 12 micron; or in other suitable ranges. Optionally, a spattering process may be utilized for fabricating the probe.

The present invention may provide impedance imaging without necessarily obtaining impedance values of sample or tissue, and may utilize impedance differences. For example, bio-impedance of tissues may be used, when a biopsy is taken several times and a false negative should be avoided; each sample may be obtained by the probe from a different tissue, in contrast with conventional methods that may attempt to indicate whether a particular tissue is cancerous or not.

In some implementations, the signal source and/or the scope may be miniaturized and may be located on the connector or interface or holding device of the probe, and may send data for processing and/or display by using a minimal set of cables or wires, or by using wireless communications (e.g., WiFi or other suitable wireless communication protocols), thereby making the probe lighter and more easily maneuverable.

In a demonstrative wireless implementation, Bluetooth communication may be used. For example, the needle connector may further comprise a microchip, signal generator, scope and a Bluetooth transmitter. Full unit miniaturization may be achieved with footprint or form-factor smaller than a mobile cellphone, thereby allowing flexible bed-side usage where the processed or raw measurements may be stored on the device or on a server side, and/or maybe displayed in real time on a local monitor or remote monitor.

The measured data and the needle model may be aligned with another imaging source or system (for example, CT scanning or ultrasound imaging), and may be aligned using markers so that the physician or nurse or probe operator may utilize augmented bio-impedance real-time imaging data.

Some embodiments of the present invention may comprise a needle system which may be a self-contained unit or autonomous unit, where the miniaturized system elements are within the connector or needle head, or implemented as part of the layers fabrication. By using microprocessors transistors fabricated on elements that are 1,000 times smaller than a hair, it may be possible to fabricate parts of the system elements of the present invention as integrated elements, and gain from lower reflections and losses of the signal as it is being transmitted and received with less steps along the way. The feedback to the user in an integrated needle may be a vibration, a sound or audio, a color indication, or other suitable output.

In some implementations, the cross-section of the tip of the needle or probe may be round; or may be non-round, for example, tapered or wedged, or may have other shape.

In some implementations, the conductive layer may not fully coat around the needle core (or around an inner insulating layer), and some inconsistency in the conductive or metallic layer may be allowed.

In some embodiments, a conductive layer may be implemented as one or more conductive wires, looped around or spiraling around the core, in a spiral or helix pattern, with or without spacing between adjacent loops; rather than being implemented as a smooth or full-surface coating layer.

The present invention may include an electrical impedance probe for spatial measurement, comprising: a needle, or other elongated shape, that is coated by multilayer conductive and insulating alternating layers; the edges of the coating layers may be distributed along the sensing area to introduce the desired spatial resolution; and at the socket side, to enable connectivity to the measurement device, a socket or interface or adapter for mounting the needle and enabling transmission of the measurement signal to the electrodes.

The needle may be hollow, to allow injection of fluid or extraction of a sample or biopsy. The needle core may be conductive, or insulating. The needle may be pre-fabricated for another use, and the probing capabilities (e.g., via multiple coating) may later be added.

The impedance-difference (or impedance contrast) positioning device of the present invention may utilize a connective socket containing electrodes, which may be connected to the different conductive layers that are coating the core needle. These electrodes may also be connected to an electrical signal transmitter (or transducer or signal source) and signal measurement system. The signal from the transducer may be transferred to and measured from each pair of neighboring or non-neighboring conductive layers, separately.

The measured data from each pair of neighboring or non-neighboring conductive layers may be analyzed in order to extract the desired information on the differences between the electrical properties of the tissues located close to different parts of the instrument. Accordingly, the current location of the instrument within different dielectric layers or materials may be determined.

In some embodiments, the system may include or may utilize, for example, one more electrode (or multiple additional electrodes) which may be located at another location, and not on the probe and not on the inserted needle; for example, the additional electrode(s) may be attached to the patient's body, and may be used for sampling of signals that are transferred to or from such additional electrode(s), for example, paired to one or more other electrodes that are located on the needle or probe itself. The system may thus analyze differences between the received signals, in order to analyze tissue or regions that may be far from the needle or probe, or not in immediate proximity to the needle or probe.

The present invention may provide an electrical impedance-difference sensor for positioning of a medical needle, and for measuring the differences between the dielectric properties of the tissues surrounding different parts of the needle. For example, the needle probe may be used as an electrical impedance-difference sensor for positioning of an epidural needle within different tissues, or within the epidural space. For example, the needle probe may be used as an electrical impedance-difference sensor for positioning of a biopsy needle within different tissues, or within a targeted tumor tissue. The present invention may be used for other purposes, for example, for energy transfer or heat transfer or heating, to be performed at a particular or desired location or depth.

In some implementations, measurements are taken and/or displayed in real time. An indication of the current position of the probe within the measured sample may be available in real time to the user of the probe. The indication may be visual, graphical, video-based, textual, acoustic or audible, or may use a combination of such indications. The indication may include information on the current sample surrounding the different parts of the needle with spatial resolution, or the information on the location of only the tip of the needle.

In some implementations, a relay unit may be introduced in or near the socket, to enable switching between the probe electrodes; or the probe may be connected directly to the signal generator and measuring system.

Optionally, the needle may be replaced by any suitable unit or structure having an elongated shape or extended shape.

In some implementations, the electric signals may be transmitted by a wireless communication device or a wireless transmitter or a wireless transceiver, which may be embedded in the socket or otherwise integrated with the socket, optionally having other suitable sub-units (e.g., a small battery; a small antenna).

It is noted that the present invention may be utilized in various fields and industries, for example, the medical industry, the cosmetics industry, the food industry, the petroleum industry, agriculture (e.g., soil analysis), or other systems which may utilized sampling of properties of layered substances.

In a first demonstrative example for a non-medical use, the present invention may be utilized for quality assurance (QA) or quality control (QC) or quality review (QR) purposes in the food industry. A probe or needle may be inserted to a food item (which may be in a solid and/or liquid form), and may measure minuscule changes in the dielectric properties to verify homogeneity of the sample, utilizing the probe's spatial resolution and ability to measure real-time properties. Since the measurement may be performed in real time in all sensing points simultaneously, a single probe may be used, for example, in order to determine the optimal time or the suitable time to begin and/or stop a particular food-related process or food-preparation step (e.g., mixing, blending, heating, cooling, or the like).

In a second demonstrative example of non-medical use, the present invention may be utilized for determining an age of a tree (or other plant) without necessarily pulling out a piece of the tree; or for obtaining information about fruit peel and/or inner structure of a fruit or a tree with a minimally-invasive technique that utilizes the probe of the present invention.

Some embodiments of the present invention may be implemented by utilizing any suitable combination of hardware components and/or software modules; as well as other suitable units or sub-units, processors, controllers, DSPs, CPUs, Integrated Circuits, output units, input units, memory units, long-term or short-term storage units, buffers, power source(s), wired links, wireless communication links, transceivers, Operating System(s), software applications, drivers, or the like.

Functions, operations, components and/or features described herein with reference to one or more embodiments of the present invention, may be combined with, or may be utilized in combination with, one or more other functions, operations, components and/or features described herein with reference to one or more other embodiments of the present invention.

While certain features of the present invention have been illustrated and described herein, many modifications, substitutions, changes, and equivalents may occur to those skilled in the art. Accordingly, the claims are intended to cover all such modifications, substitutions, changes, and equivalents.

1-46. (canceled)

47. A system for spatial impedance imaging, comprising:
a multi-layer coaxial probe for spatial impedance imaging, comprising:
an elongated core having a distal end and a proximal end;
a first coating layer wrapping around the core;
a set of alternating conductive and insulating coating layers on top of said first coating layer, wherein an Nth coating layer is shorter than an N-1th coating layer beneath it;
wherein each coating layer begins at a first distance from the proximal end, and ends at a second distance from the distal end; wherein values of the first distance and the second distance increase for external coating layers relative to internal coating layers.

48. The system of claim 47, wherein each coating layer has a thickness of approximately 0.5 micron to 50 micron; wherein a tip of the probe comprises a tip selected from the group consisting of: a round tip, a tapered tip.

49. The system of claim 47, wherein at least one of the coating layers comprises a partial coating layer that provides partial coating to a layer underneath said partial coating layer.

50. The system of claim 47, wherein at least one pair of an adjacent conductive coating layer and insulating coating layer comprises:
a conductive coating layer formed of a metal; and
an insulating coating layer formed of said metal that was subjected to oxidation.

51. The system of claim 47, wherein edges of the coating layers are distributed along a sensing area adjacent to a tip of said probe in accordance with a desired spatial resolution.

52. The system of claim 47, wherein the core comprises a hollow needle to enable at least one of: injection of a fluid; delivery of a drug; extraction of a biopsy;
wherein the hollow needle within the core comprises a pre-fabricated medical needle that is subsequently coated with said alternating coating layers.

53. The system of claim 47, further comprising:
an electric signal source to provide an electric signal to each pair of conductive layers;
an electric signal measurement unit to measure impedance differences between pairs of conductive layers;
a processing module to determine a location of a tip of said multi-layer coaxial probe within a sampled item, based on said measured impedance differences;
an output unit to provide to a user of the probe a real-time indication of a current location of a tip of the probe; wherein the real-time indication comprises at least one of: an audible indication, a vibrating indication, a visual indication;
wherein the probe is connected to a socket comprising a set of electrodes to receive an electromagnetic signal via electric wires from a signal source.

54. The system of claim 53, wherein said pairs of conductive layers comprise at least one pair of non-neighboring conductive layers.

55. The system of claim 53, wherein said pairs of conductive layers comprise at least one pair of neighboring conductive layers.

56. The system of claim 47, wherein the set of alternating coating layers coat a tip of said probe to form therein one or more sensing points.

57. The system of claim 47, wherein the conductive layers are formed of titanium, and wherein the insulating layers are formed of glass.

58. The system of claim 47, wherein the probe is directly connected to a signal generator and a measuring sub-system.

59. The system of claim 47, wherein the probe is indirectly connected via a socket, to a signal generator and a measuring sub-system;
wherein the socket comprises a relay module to enable switching among electrodes of the probe;
wherein the socket comprises: a battery, and a wireless transmitter to wirelessly transmit electric signals.

60. The system of claim 47, wherein the elongated core comprises an element selected from the group consisting of:
an elongated needle-like member;
an epidural needle;
a biopsy needle;
a drug delivery needle;
a cosmetic needle;
an intravenous (IV) needle;
a draining needle;
a needle having a non-circular cross-section;
a needle having a triangular cross-section;
a needle having a square-shaped cross-section;
a thin sharp blade;
a heat-transfer unit to enable selective heating of a location of interest;
an electrical energy-transfer unit to enable selective electric stimulation of a location of interest.

61. The system of claim 47, wherein the multi-layer coaxial probe is a multi-layer quasi-coaxial probe which is non-symmetric along the long dimension of the elongated core.

62. The system of claim 47, wherein each coating layer has a thickness in the range of 0.1 percent to 3 percent of a thickness of the elongated probe.

63. The system of claim 47, wherein at least one of the conductive coating layers comprises:
a first layer formed of a first metal, having thickness of 1 to 300 nanometers; covered by
a second layer formed of a second metal, having thickness of 500 to 5,000 nanometers, said second metal having greater electrical conductivity than said first metal; covered by
a third layer formed of said first metal, having thickness of 1 to 300 nanometers.

64. The system of claim 47, wherein at least one of the conductive coating layers comprises:
a first layer of titanium; covered by a layer of a metal other than titanium, said metal having greater electrical conductivity than titanium; covered by
a second layer of titanium.

65. The system of claim 47, wherein at least one of the conductive coating layers comprises:
a first layer of titanium, having thickness of 1 to 250 nanometers; covered by
a layer of a metal other than titanium, having thickness of 500 to 5,000 nanometers, said metal having greater electrical conductivity than titanium; covered by
a second layer of titanium, having thickness of 1 to 250 nanometers.

66. The system of claim 47, wherein an outmost coating layer of said multi-layer coaxial probe is formed of a material selected from the group consisting of:
a bio-compatible material;
a hydrophobic material;
a hydrophilic material;
a gold layer;
a gold layer applied by electro-plating.
